(19) United States
(12) Patent Application Publication
(10) Pub. No.: US 2002/0177573 A1
(43) Pub. Date: Nov. 28, 2002

(54) Exotoxin-ligand

(76) Inventors: Hans E. Maschke, Bad Homburg (DE); Veit Otto, St. Wendel (DE)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/113,809

(22) Filed: Mar. 29, 2002

(30) Foreign Application Priority Data
Mar. 30, 2001 (DE) 101 16 042.9-41

(51) Int. Cl. A61K 39/395; A61K 48/00

(52) U.S. Cl. 514/44; 424/140.1; 514/14

(57) ABSTRACT

The present invention relates to a ligand for bacterial toxins, particularly entero- or exotoxins of gram-positive bacteria, which is capable of selective interaction with a structure containing an amino acid sequence conserved in the bacterial toxins. The invention also relates to an adsorbent that exhibits the ligand bound to a matrix, an adsorption device for reducing the concentration of bacterial toxins in blood or blood plasma as well as a pharmaceutical composition, which contains the ligands, and which is suitable in particular for the treatment and/or prevention of gram-positive sepsis.

The present invention relates to a ligand for bacterial toxins, especially entero- or exotoxins of gram-positive bacteria, which is capable of selective interaction with a structure containing an amino acid sequence conserved in the bacterial toxins. The invention also relates to an adsorbent that exhibits the ligand bound to a matrix, an adsorption device for reducing the concentration of bacterial toxins in blood or blood plasma as well as a pharmaceutical composition, which contains the ligands, and which is suitable in particular for the treatment and/or prevention of gram-positive sepsis.

Bacterial superantigens are among the strongest toxins. To the superantigen family belong, for example, the entero- or exotoxins of gram-positive bacteria, such as SEA through SEE (SEB being the most frequent) from Staphylococcus, toxic-shock syndrome toxin 1 (TSST-1), as well as SPEA and SPEC from Streptococcus. These induce, by bonding to the T-cell receptor (TCR) and/or the Major Histocompatibility Complex II (MHC II) of the T-helper cells (Th1), the production and distribution of lymphokines (or cyctokines or interleukins) such as, for example, IL-2, IFN-γ and/or TNF-β. These molecules act as superantigens uncoupled from the normal mechanism for the activation of the immune response via the presentation of processed antigens.

Materials that remove these pyrogenically acting substances from the blood have, among other things, been proposed according to the state of the art for the treatment or prevention of toxic shock triggered by such superantigens. Thus, in U.S. Pat. No. 4,381,239, agents for the adsorption of pyrogens were described, which include an insoluble carrier and a heterocyclic compound containing nitrogen. Disclosed in U.S. Pat. No. 5,928,633 is a material containing a urea- or thiourea-bond, which exhibits an affinity for Staphylococcus enterotoxin and Streptococcus exotoxin. Described in DE-A 197 05 366, furthermore, is a device for the purification of protein-containing solutions, such as blood, blood plasma or culturing media, which includes a biologically compatible carrier material consisting of plastic covalently coated with albumin via peptide bonds and capable as a result of binding a series of toxins, for example, exogenic toxins.

The ligands contained in those absorbent agents known to the state of the art bind however relatively unspecifically to the most diverse points on the protein structures to be bound and/or exhibit an affinity merely to individual representatives of the superantigens.

The present invention thus addresses the problem of making a new system for binding bacterial toxins available, which is based upon the selective interaction with those structures preserved in the toxins and therefore usable in pharmaceutical compositions and absorbent agents in order, for example, to act or prevent a septic shock induced by or to be feared from such bacterial toxins.

This problem is solved by those embodiments of the present invention characterized in the claims.

In particular, a ligand for bacterial toxins is provided, which is capable of selective interaction with the β-sheet-hinge-α-helix structure of the superantigens.

The expression “capable of selective interaction” means that the ligand of the present invention has a high affinity to the structure conserved in the toxins and therefore interacts with it preferentially. By preference, the association constant of the ligand for the toxin or structure amounts, for example, in vitro under physiological or substantially physiological conditions, such as at 37° C., pH 7.4 and approximately from 140 to 150 mM of NaCl, to at least 10⁶ M-¹, more preferably at least 10⁸ M⁻¹. The interaction of the ligand can thereby include any type of chemical or physical bond, including, for example, electrostatic interactions, Van der Waals interactions, hydrogen-bridge bonds and/or hydrophobic interactions.

The provision of the ligand of the present invention is thus based upon the insight that those bacterial toxins included among the superantigens exhibit only slight homology with one another with regard to their overall sequences, though conserved sequence motifs are present within these sequences or conserved spatial structures are formed by these coherent amino acid sequences and/or by amino acid residues lying at a distance from one another in the linear sequence.

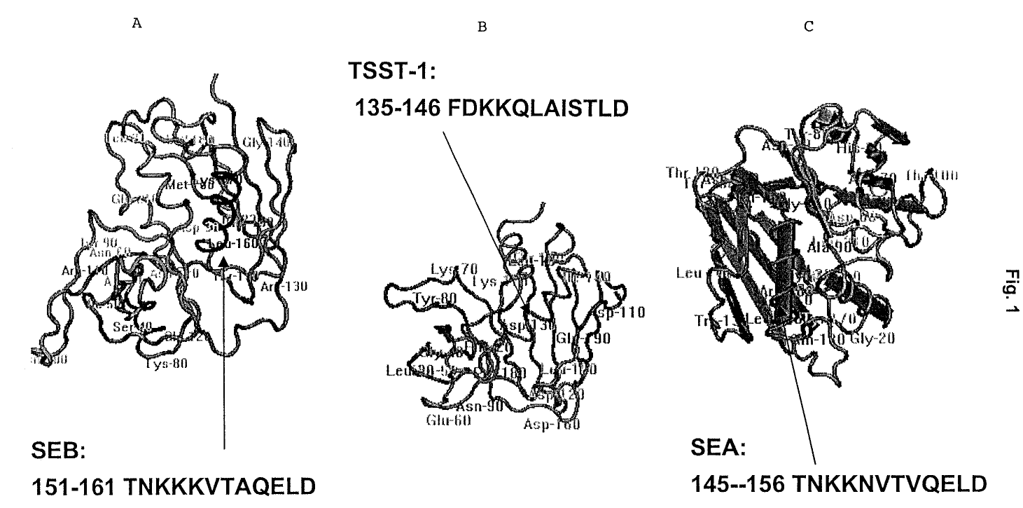

In particular, the β-sheet-hinge-α-helix structure of the superantigens, also designated as P12, is strongly conserved (compare FIG. 1) and generally includes a dodecapeptide. The corresponding sequence of SEB includes amino acids 150 through 161 and is read as TNKKKVTAQELD (SEQ ID NO. 1). The corresponding structural motif in SEA (TNKKNVTVQELD, amino acids 145 through 156, SEQ ID NO. 2) differs in only two positions from that of SEB. The corresponding sequence area of TSST-1 (FDKKQLAISTLD, amino acids 135 through 146, SEQ ID NO. 3) exhibits of course only four identical radicals when compared with SEB, but forms a very similar spatial structure, as shown by the formation marked in red in FIG. 1. The above amino acid numbering refers to those sequences published by Papageorgiou et al. (Protein Sci. 5(8), 1737-1741, 1996), Sundstrom et al. (J. Biol. Chem. 271, 32212-32216, 1996) or Papageorgiou et al. (J. Mol. Biol. 277, 61-79, 1998).

A possible significance of this conserved structure for the study and mode of operation of the bacterial toxins in question was pointed out in Arad et al. Nature Medicine 6, 414-421, 2000. According to that, the dodecapeptide YNKKKVTAQELD (SEQ ID NO. 4), in which, in comparison with the starting sequence-motif of SEB, the first threonine was replaced by tyrosine, inhibits the activation of the immune response, i.e. of lymphokine production, as an antagonist, because the corresponding first amino acid in the case of TSST-1 is a phenylalanine. By intravenous administration of the peptide, it was possible to inhibit septic shock in an animal experiment employing mice. The inhibition of lymphokine production with regard to the immune induction modulated by SEA, SEB, TSST-1 and SPEA was also thereby in evidence. In any case, Arad et al. did not indicate the possibility of producing a ligand directed against a structure conserved in the bacterial toxins, for example, β-sheet-hinge-α-helix domain.

By preference, the ligand of the present invention thus binds exo- or enterotoxins of gram-positive bacteria, such as SEA, SEB, SEC, SED, SEE, TSST-1, SPEA and SPEC.

According to preferred embodiments, the ligand of the present invention can be a synthetic organic ligand, a saccharide, for example, an oligosaccharide, a peptide, for example, an oligopeptide, or a nucleic acid such as a single-stranded oligonucleotide. An example of a peptide ligand according to the invention is an antibody directed against the conserved structure of the superantigens. The antibody can be thereby monoclonal, polyclonal or recombinant, or it can be a chimaera, consisting, for example, of a variable mouse component and an invariant component from man.

With regard to possible immunological problems, particularly when the ligand of the present invention is contained in an absorbent agent, it is preferred for the ligand to include an oligopeptide, oligosaccharide or oligonucleotide, in which case such oligomers are more preferably those with not more than 100 components.

From a further point of view, the present invention relates to methods for the production of the ligand of the present invention.

Various procedures can be employed to produce the ligand of the present invention, in which case consideration must be given to the following points during selection:

the thermal stability of the ligands as well as stability relative to the biological environment, (for example, blood or blood plasma),

the great variability of the ligands for covering a large target molecule group during ligand identification,

the economy of the process, and

affinity of the ligands for the target structure adequate for it to be used in the absorption from blood or plasma.

It is thus for example possible to identify synthetic ligands by the use of so-called SPOT synthesis. SPOT synthesis (or stain synthesis) offers the possibility of producing peptide libraries on solid phases rapidly and flexibly by automated parallel synthesis. The principle consists of the distribution of extremely small droplets on zones that have been precisely defined beforehand with solutions of reagents (English: “spots”, stains) on a suitable surface (for example, cellulose membranes, glass, etc.). Fixing on a shared surface offers the advantage that the resulting libraries will be present in an easily manageable format. The intermediate steps in the synthesis, such as washing or the removal of protective groups, can be carried out in common for all members of the library. In addition to that, the library in this form can also be screened in common, which as a whole results in a clear saving of time and material.

The SPOT technique can be employed for the construction of the most diverse ligand systems by the use of a modular system developed by Jerini et al., supra. The pilot structure to be bound, for example, a structure containing the amino acid sequence TNKKKVTAQELD SEQ. ID NO. 1, can be optimized or stabilized according to need. In addition to carrying out mutations with the natural amino acids, it is possible to introduce any desired component at any position in the sequence. An extensive repertoire of organic-synthetic components are available, which are known to the expert. For example, 1,3,5-chlorotriazine components can be used, whose introduction makes numerous modification possibilities available via the possibility for multiple substitution. By means of the stepwise substitution of all amino acids, it is also possible to produce a completely synthetic organic ligand. These facts are made clear by the following reaction diagram.

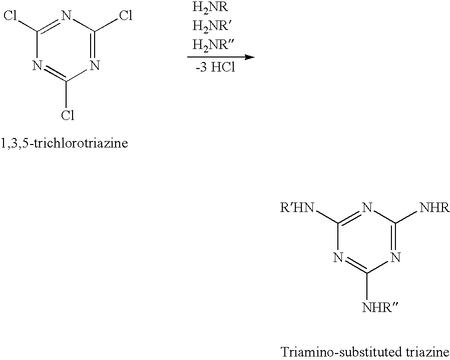

Such synthetic ligands are stable in a biological environment such as blood and blood plasma.

It should be understood that, the desired peptide can per se be chemically modified or derived by means of methods known to the state of the art.

A further method for producing the peptide-based ligand of the present invention makes use of the so-called phage-display technique. An additional peptide sequence is thereby presented on the surface of a phage by manipulation of the DNA of this phage and thus accessible for binding tests with the target molecule. If a sequence that binds the target molecule is detected in the phage library used, its primary structure can be clarified in the known manner by amplification of the corresponding phage-DNA and its sequencing. An advantage of the phage-display technique consists for example of the fact that an initially identified ligand can be optimized with this technique in a simple manner relative to its affinity for the target molecule. For that purpose, the nucleotide sequence coding for the peptide ligand is modified (mutagenesis) by the exchange, addition, deletion and/or insertion of one or more nucleotides in such a way that, starting from the initially identified peptide ligand, a ligand with improved affinity for the target structure is made available rapidly and simply, which exhibits an amino acid sequence modified according to the manipulation of the coding DNA.

One problem with the original phage-display technique consists of the fact that, similar to the case with combinatorial peptide libraries, libraries in excess of a certain number of amino acids can be built up only with difficulty. (A library of peptide decamers on the basis of all 20 naturally occurring amino acids requires, for example, more than 10¹⁵ clones.) The utilization of shorter peptides does not however as a rule achieve the binding affinities of relatively long peptides. Therefore, according to the invention, use is made preferably of the “Cosmix strategy” (Cosmiplexing Technique, Cosmix Molecular Biologicals GmbH): In a first selection cycle, a preliminary selection of peptide sequences that interact with the target structure takes place. An optimization of the affinity for the target molecule is then carried out, by means of the cosmiplexing technique, within those sequences found. The binding optimization takes place particularly effectively, because an extremely high diversity is achieved in the preselected sequence library via the specific combination technique employed, leading to an optimal combination of binding structure increments. This leads to affinities, particularly within short peptide sequences, which are otherwise achieved only with oligomers having relatively high molecular weight from significantly larger basic libraries.

According to another embodiment, the multiplicity of phage-display libraries can be restricted by limiting the mutation-capable starting sequence. Protease inhibitors are for example made available as the basis for constructing a phage-display library by the Dynax Company (Cambridge, Mass., USA). Parts of these proteins are presented on the phages in the library, and mutations of one or more amino acids are undertaken within the binding domains. The problem of the possible instability in biological fluids such as blood or blood plasma, arising with the use of natural peptides, can be avoided by the use of ligands on a protein-inhibitor basis, which generally exhibit high biological stability.

For screening, the target molecule is immobilized on microspheres or beads and brought into contact with the phage library in the known manner. The microbeads are isolated with the phages bound in them via the peptide ligands, and the phages are cleaved enzymatically from the peptides they present. The phages thus separated, which contain the DNA molecule containing the peptide ligands coding for the target molecule, are amplified in E. coli and thus available for additional steps, for example, the mutagenesis cited above for optimization of the affinity of the ligand with regard to the target structure.

Furthermore, nucleic acids can also be employed, on the basis of specific protein-nucleic acid interactions (for example, protein/RNA in ribosomes and protein/DNA in nucleases) frequently occurring in nature, for the production of the ligands of the present invention. The production of a large number of sequence and thus structure variants is easily possible with the use of nucleic acids via a base substitution. Sequences with a predefined length are coupled to a matrix and, as described above relative to phage displays, brought into contact with the target molecule. Binding nucleic acids thereby exhibit a structure capable of interaction with the target molecule. As for optimization of the sequence for preparation of a ligand with relatively high affinity, nucleic acids exhibit the advantage that they can be easily amplified (for example, in vitro, by PCR or in vivo using E. coli), for which reason the binding of a single nucleic acid molecule can suffice as a starting point for binding optimization. Under suitable conditions, an additional passage of an enriched (amplified) initial nucleic acid mixture can be carried out as described above. As in the case of those peptide ligands described above, the problem of instability in a biological environment can also occur with nucleic acids. In particular, RNA is frequently less stable than DNA. Single-stranded as well as double-stranded species enter into consideration as nucleic acid ligands (in which case intramolecular base pairs can obviously also occur in a single-stranded molecule). Suitable for stabilization against nucleases naturally occurring in biological fluids such as blood or blood plasma are, for example, those procedures described below.

A preferred method for the production of a ligand of the present invention on a nucleic acid basis is based upon the principle of the so-called mirror-image technology (compare WO 98/00885). In that case, a “mirror image” of the target molecule (for example, the dodecapeptide with the SEQ ID NO. 4) is synthesized, i.e. its enantiomer, consisting of L-amino acid components. The target molecule, mirrored in this way, is then screened with a library consisting for example of RNA-oligomers in their natural D-form. The sequence of binding library members is determined and then synthesized in the form of its L-isomers, which do not occur naturally. These L-isomers bind to the unmirrored target molecule on the basis of stereochemistry. Nucleic-acid ligands are thus prepared, which are particularly suitable for use in biological fluids, because they are essentially biologically inert as a result of the L-form not occurring in nature.

Ligands on the basis of nucleic acid can furthermore be stabilized by the incorporation of phosphate-modified nucleotides. Stabilization in a biological environment is thereby achieved by the incorporation of nucleotides correspondingly modified, for example, via PCR. Possible, for example, are substitutions of oxygen in the phosphate group by sulfur (compare ³⁵S-DNA sequencing, α-thiophosphates) or by a methyl group. Such modified ssDNA molecules are protected, for example, from degradation by DNAases.

A further possibility for the stabilization in particular of DNA-ligands consists of methylation using methylases. Thus, for example, DNA-ligands can be produced in bacteria such as E. coli in the presence of various methylases, which are preferably present in plasmid-coded form. Double-stranded segments produced in ssDNA-ligands by intramolecular base pairing, in particular, are thereby protected by methylation against endonucleases. An effective protection against exonucleases can be achieved in addition by the attachment of repetitive “cap” sequences.

Application possibilities for the use of the ligand defined above include, for example, its use for the purification of bacterial toxins or their removal from fluids such as, for example, blood or blood plasma, and for the inhibition of target bacterial toxins on the basis of selective binding to a conserved structure present therein.

A further object of the present invention therefore relates to an adsorbent comprising a matrix, preferably an organic matrix, and at least one above-defined ligand covalently bound to the matrix.

The adsorbent of the present invention is preferably biologically compatible. A “biologically compatible” adsorbent is preferably blood- or plasma-compatible. According to a further preferred embodiment, it is compatible with whole blood.

In principle, several carrier materials are conceivable as a matrix, such as, for example, glass, carbohydrates, Sepharose®, silica or organic matrices, such as copolymers of acrylates or methacrylates as well as polyamides. The matrix consists preferably of organic material and more preferably of copolymers derived from (meth)acrylic acid esters and/or amides. These preferably exhibit epoxide groups. To be understood by the term “(meth)acrylic” are both the corresponding acrylic and methacrylic compounds.

Most preferred as a matrix for the adsorbent of the present invention agent is a statistical copolymer produced by polymerization of the monomeric units:

(A) (Meth)acrylamide in a quantity of from 10 to 30% by weight,

(B) N,N-methylene-bis(meth)acrylamide in a quantity of from 30 to 80% by weight, and

(C) Allylglycidyl ether and/or glycidyl (meth)acrylate in a quantity of from 10 to 20% by weight, respectively with regard to the total weight of the monomeric units.

The copolymer is produced preferably by suspension polymerization.

Such a copolymer is available commercially under the designation Eupergit C250L or Eupergit FE162 from Rohm GmbH.

With the use of the above-named copolymer or of another organic matrix containing oxirane (epoxide) groups, for example, a copolymer preferred likewise within the context of the present invention, obtained by suspension polymerization of ethylene glycol dimethacrylate and glycidyl methacrylate and/or allylglycidyl ether, these oxirane groups are aminated prior to the introduction of the ligand to be covalently bound, preferably with ammoniac or a primary amine. Ammoniac is thereby preferred for reasons having to do with process technology and cost.

When the adsorbent of the present invention is utilized for the removal of bacterial toxins, such as entero- or exotoxins of gram-positive bacteria from blood or blood plasma, the matrix can be present for example in the form of spherical, unaggregated particles, so-called microspheres or beads, fibers or a membrane, a porous matrix thus being prepared, which exhibits a relatively large surface. The formation or adjustment of porosity can be achieved for example by the addition of pore-forming agents such as cyclohexanol or 1-dodecanol to the suspension-polymerization reaction mixture for the matrix. It is further advantageous for the matrix to possess an exclusion threshold of at least 10⁷ daltons, so that the bacterial toxins can penetrate into the pores with the blood, in order to reach the matrix-bound ligands.

A further advantageous embodiment of the invention lies in the application of the adsorbent of the present invention in whole blood via an appropriate choice of carrier matrix. The matrix will thereby consist of unaggregated spherical particles in a particle-size range of from 50 to 250 μm and possesses an exclusion boundary of at least 10⁷ daltons. As a result, blood cells can enter into contact with the adsorbent material, without the column becoming clogged or an unreasonably large number of cells being held back or aggregating. This is made possible by the size and the spherical shape of the beads in conjunction with the exclusion threshold of the adsorbent of the present invention, because the cells glide along the smooth outer surface of the beads, thereby resulting in only slight thrombocyte adhesion, which nevertheless permits the plasma to penetrate into the pores with the bacterial toxins.

This eliminates extracorporeal steps, such as the separation of blood cells, the treatment of the isolated plasma and subsequent bringing together of the blood components, increasing the biological compatibility of the process, which further considerably reduces for example the danger of complement activation. The elimination of extracorporeal steps results furthermore in a reduction of treatment time and a simplification of the process, thus permitting the most rapid possible removal of the toxins from the patient's blood circulation.

The adsorbent of the present invention can furthermore be utilized for the purification of bacterial toxins, preferably entero- and exotoxins of gram-positive bacteria.

A process for the purification of bacterial toxins from a fluid is also therefore made available according to the invention, which includes the steps:

a) Providing the adsorbent as defined above, and

b) Contacting the fluid with the adsorbent.

The purification process according to the invention is preferably carried out continuously, with the adsorbent provided for example in a chromatographic column and the fluid with the toxins to be purified being added in the known manner. It is likewise possible, however, to employ the adsorbent of the present invention in a batch process.

As stated above, an adsorbent containing the ligand of the present invention for bacterial toxins can serve to reduce the concentration of bacterial toxins, especially of entero- and exotoxins of gram-positive bacteria in blood or blood plasma. For that purpose, the adsorbent according to the invention is employed during production of the corresponding adsorption device.

A further object of the present invention thus relates to an adsorption device for reduction of the concentration of bacterial toxins in blood or blood plasma, which exhibits a housing preferably in the form of a tube or column, with the adsorbent as the filling material. In view of the quantities of blood or blood plasma to be passed through and the efficiency of the adsorption device of the present invention, the latter will preferably exhibit a volume of from 30 to 1,250 ml, more preferably from 50 to 200 ml, particularly when the unit is a regenerating adsorption device. The adsorption device can be employed in single, double or multiple operation. The use of two or more adsorption devices provides the option of alternatingly charging one adsorption device with blood or plasma, while the other adsorption device is being regenerated. This leads to a further increase in efficiency during use of the adsorption device of the present invention, particularly because it can be crucial during the treatment and/or prevention of a gram-positive sepsis with an adsorption device to remove the toxins in question from the patient's blood or blood plasma as rapidly as possible. The adsorption device is preferably designed in such a way that it exhibits a housing with an inlet area at the top, through which the blood plasma is introduced, the outlet being in this case located at the bottom of the housing.

A filter is preferably arranged at the outlet of the housing of the adsorption device to prevent unwanted substances, for example, substances originating from the adsorbent material, from being passed back into the patient's blood circulation along with the treated blood or blood plasma. The filter is preferably a particle filter.

With the use of the adsorption device of the present invention, a process is also made available for the treatment and/or prevention of gram-positive sepsis, in which the blood or blood plasma of a patient infected with the bacteria in question, such as Staphylococcus or Streptococcus, where septic shock is to be feared on the basis of the toxins originating from the bacteria, is passed in a circuit over the adsorption device of the present invention.

As explained above, the ligand of the present invention can also be employed, by virtue of its selective binding to the concerned bacterial toxins, for the inhibition or reduction of the toxic effects of these molecules.

According to the invention, a pharmaceutical composition is therefore provided, which contains the ligand of the present invention as well as, if necessary, one or more pharmaceutically acceptable carriers and/or diluents. The pharmaceutical composition according to the present invention is preferably employed for the treatment and/or prevention of gram-positive sepsis. The pharmaceutical composition can preferably be administered for that purpose to the patient, for example, by oral, intravenous, intramuscular, subcutaneous and/or topical means. The intravenous administration of the pharmaceutical composition can thereby include a bolus injection and/or a continuous infusion of an effective quantity of the ligand of the present invention.

A further application possibility for the ligand of the present invention consists of its use for detection of the bacterial toxins in question, which can serve for example to trace an infection possibly occurring due to an infection with bacteria, such as gram-positive bacteria, to permit suitable therapeutic countermeasures to be established at an early point in time.

According to a further point of view, a diagnostic kit is provided, containing the ligand of the present invention, which preferably exhibits one or more detectable label(s).

The expression “detectable labels” relates to any labels known to the expert active in this field, which include radioactive labels, covalently or not covalently bonded to the ligand, one or more dyes that absorb light in the visible range, fluorescent dyes, such as fluorescein, fluorescein isothiocyanate (FITC), Texas red and fluorescent dyes from the cy-series, biotin, digoxigenin, etc.

The kit of the present invention is preferably employed for detection of a sepsis with gram-positive bacteria that exists or is to be feared.

The figures show:

FIG. 1 is a graphic illustration showing the peptide backbone of the spatial structures of the superantigens SEB (FIG. 1A), TSST-1 (FIG. 1B) and SEA (FIG. 1C). The amino acid sequences (SEQ ID NO. 1 through 3) of the conserved β-sheet-hinge-α-helix domains and their position in the overall sequence are indicated.

1. Ligand for bacterial toxins, which is capable of selective interaction with the β-sheet-hinge-α-helix structure of the superantigens.

2. Ligand according to claim 1, wherein the toxin is an entero- or exotoxin gram-positive bacteria.

3. Ligand according to claim 2, wherein the toxin is selected from the group consisting of SEA, SEB, SEC, SED, SEE, TSST-1, SPEA and SPEC.

4. Ligand according to any one of claims 1 to 3, wherein the structure includes the amino acid sequence YNKKKVTAQELD (SEQ ID NO. 4).

5. Ligand according to any one of claims 1 to 4, which contains an oligosaccharide and/or an oligopeptide and/or an oligonucleotide.

6. Adsorbent, containing a matrix and at least one ligand according to any one of the claims 1 to 5, covalently bound to the matrix.

7. Adsorbent according to claim 6, wherein the matrix is an organic matrix.

8. Adsorbent according to claim 7, wherein the organic matrix is a copolymer derived from (meth)acrylic acid esters and/or amides.

9. Adsorbent according to claim 8, wherein the copolymer derived from (meth)acrylic acid esters and/or amides contains epoxide groups.

10. Adsorbent according to claim 8 or 9, wherein the copolymer is a statistical copolymer produced by polymerization of the monomer groups:
(A) (Meth)acrylamide in a quantity of from 10 to 30% by weight,
(B) N,N-methylene-bis(meth)acrylamide in a quantity of from 30 to 80% by weight, and
(C) Allylglycidyl ether and/or glycidyl (meth)acrylate in a quantity of from 10 to 20% by weight, respectively with regard to the total weight of the monomeric units.

11. Adsorbent according to any one of claims 8 to 10, wherein the epoxide groups are aminated with ammoniac or a primary amine before introduction of the side chains.

12. Adsorbent according to any one of claims 7 to 11, wherein the organic matrix consists of spherical, unaggregated particles.

13. Adsorbent according to claim 12, wherein the spherical, unaggregated particles exhibit a particle size of from 50 to 250 μm.

14. Adsorbent according to any one of claims 7 to 13, wherein the organic matrix exhibits an exclusion boundary of at least 10⁷ daltons.

15. Adsorbent according to any one of claims 6 to 14, wherein the adsorbent is biologically compatible.

16. Adsorbent according to any one of claims 6 to 15, wherein the adsorbent is compatible with whole blood.

17. Use of the adsorbent according to any one of claims 6 to 19 for the purification of bacterial toxins.

18. The use according to claim 17, wherein the toxins are entero- or exotoxins of gram-positive bacteria.

19. A process for the removal of bacterial toxins from a fluid, comprising the steps:
(a) Providing the adsorbent according to any one of claims 6 to 16, and
(b) Contacting the fluid with the adsorbent.

20. The process according to claim 19, wherein the toxins are entero- or exotoxins of gram-positive bacteria.

21. Use of the adsorbent according to any one of claims 6 to 16 for the production of an adsorption device for reducing the concentration of bacterial toxins in blood or blood plasma.

22. The use according to claim 21, wherein the toxins are entero- or exotoxins of gram-positive bacteria.

23. Adsorption device for reducing the concentration of bacterial toxins in blood or blood plasma, consisting of a housing and of the adsorbent according to any one of claims 6 to 16 contained in the housing.

24. Adsorption device according to claim 23, wherein the adsorption device comprises a volume of from 30 to 1,250 ml.

25. Adsorption device according to claim 24, wherein the adsorption device exhibits a volume of from 50 to 200 ml.

26. Adsorption device according to claim 25, wherein the adsorbent can be regenerated.

27. Adsorption device according to any one of claims 23 to 26, wherein the adsorption device exhibits an inlet area at the top and an outlet area at the bottom.

28. Adsorption device according to any one of claims 23 to 27, wherein the adsorption device exhibits a filter arranged in its outlet area.

29. Adsorption device according to claim 28, wherein the filter is a particle filter.

30. Pharmaceutical composition, containing ligands according to any one of claims 1 to 5, optionally in conjunction with one or more pharmaceutically acceptable carrier(s) and/or diluent(s).

31. Pharmaceutical composition according to claim 30 for the treatment and/or prevention of gram-positive sepsis.

32. Diagnostic kit, containing the ligand according to any one of claims 1 to 5.

33. Kit according to claim 32, wherein the ligand exhibits one or more labels.

34. Kit according to claim 32 or 33 for the detection of sepsis with gram-positive bacteria.
